SEQUENCE LISTING (28 sequences)

SEQ ID NO: 1 - RNA, 23 nt, Homo sapiens
uuaaugcuaaucgugauaggggu

SEQ ID NO: 2 - RNA, 22 nt, Homo sapiens
cuccuacauauuagcauuaaca

SEQ ID NO: 3 - DNA, 16 bp, Artificial Sequence
  oligonucleotide inhibitor of miR-155-5p
  misc_feature (1)..(16): May be joined through phosphorothioate bonds
  modified_base (1)..(1): May be locked nucleic acid adenosine
  modified_base (5)..(5): May be locked nucleic acid cytidine
  modified_base (6)..(6): May be locked nucleic acid guanosine
  modified_base (8)..(8): May be locked nucleic acid thymidine
  modified_base (10)..(10): May be locked nucleic acid adenosine
  modified_base (11)..(11): May be locked nucleic acid guanosine
  modified_base (13)..(13): May be locked nucleic acid adenosine
  modified_base (15)..(15): May be locked nucleic acid thymidine
  modified_base (16)..(16): May be locked nucleic acid adenosine
atcacgattagcatta

SEQ ID NO: 4 - DNA, 16 bp, Artificial Sequence
  oligonucleotide inhibitor of miR-155-5p
  misc_feature (1)..(16): May be joined through phosphorothioate bonds
  modified_base (1)..(1): May be locked nucleic acid adenosine
  modified_base (5)..(5): May be locked nucleic acid cytidine
  modified_base (6)..(6): May be locked nucleic acid guanosine
  modified_base (9)..(9): May be locked nucleic acid thymidine
  modified_base (10)..(10): May be locked nucleic acid adenosine
  modified_base (11)..(11): May be locked nucleic acid guanosine
  modified_base (13)..(13): May be locked nucleic acid adenosine
  modified_base (15)..(15): May be locked nucleic acid thymidine
  modified_base (16)..(16): May be locked nucleic acid adenosine
atcacgattagcatta

SEQ ID NO: 5 - DNA, 16 bp, Artificial Sequence
  oligonucleotide inhibitor of miR-155-5p
  misc_feature (1)..(16): May be joined through phosphorothioate bonds
  modified_base (1)..(1): May be locked nucleic acid adenosine
  modified_base (2)..(2): May be locked nucleic acid thymidine
  modified_base (6)..(6): May be locked nucleic acid guanosine
  modified_base (8)..(8): May be locked nucleic acid thymidine
  modified_base (10)..(10): May be locked nucleic acid adenosine
  modified_base (11)..(11): May be locked nucleic acid guanosine
  modified_base (13)..(13): May be locked nucleic acid adenosine
  modified_base (15)..(15): May be locked nucleic acid thymidine
  modified_base (16)..(16): May be locked nucleic acid adenosine
atcacgattagcatta

SEQ ID NO: 6 - DNA, 16 bp, Artificial Sequence
  oligonucleotide inhibitor of miR-155-5p
  misc_feature (1)..(16): May be joined through phosphorothioate bonds
  modified_base (1)..(1): May be locked nucleic acid adenosine
  modified_base (2)..(2): May be locked nucleic acid thymidine
  modified_base (6)..(6): May be locked nucleic acid guanosine
  modified_base (7)..(7): May be locked nucleic acid adenosine
  modified_base (10)..(10): May be locked nucleic acid adenosine
  modified_base (11)..(11): May be locked nucleic acid guanosine
  modified_base (12)..(12): May be locked nucleic acid cytidine
  modified_base (14)..(14): May be locked nucleic acid thymidine
  modified_base (16)..(16): May be locked nucleic acid adenosine
atcacgattagcatta

SEQ ID NO: 7 - DNA, 16 bp, Artificial Sequence
  oligonucleotide inhibitor of miR-155-5p
  misc_feature (1)..(16): May be joined through phosphorothioate bonds
  modified_base (1)..(1): May be locked nucleic acid adenosine
  modified_base (5)..(5): May be locked nucleic acid cytidine
  modified_base (6)..(6): May be locked nucleic acid guanosine
  modified_base (8)..(8): May be locked nucleic acid thymidine
  modified_base (10)..(10): May be locked nucleic acid adenosine
  modified_base (11)..(11): May be locked nucleic acid guanosine
  modified_base (13)..(13): May be locked nucleic acid adenosine
  modified_base (14)..(14): May be locked nucleic acid thymidine
  modified_base (16)..(16): May be locked nucleic acid adenosine
atcacgattagcatta

SEQ ID NO: 8 - DNA, 16 bp, Artificial Sequence
  oligonucleotide inhibitor of miR-155-5p
  misc_feature (1)..(16): May be joined through phosphorothioate bonds
  modified_base (1)..(1): May be locked nucleic acid adenosine
  modified_base (2)..(2): May be locked nucleic acid thymidine
  modified_base (5)..(5): May be locked nucleic acid cytidine
  modified_base (9)..(9): May be locked nucleic acid thymidine
  modified_base (10)..(10): May be locked nucleic acid adenosine
  modified_base (12)..(12): May be locked nucleic acid cytidine
  modified_base (13)..(13): May be locked nucleic acid adenosine
  modified_base (15)..(15): May be locked nucleic acid thymidine
  modified_base (16)..(16): May be locked nucleic acid adenosine
atcacgattagcatta

SEQ ID NO: 9 - DNA, 16 bp, Artificial Sequence
  oligonucleotide inhibitor of miR-155-5p
  misc_feature (1)..(16): May be joined through phosphorothioate bonds
  modified_base (1)..(1): May be locked nucleic acid adenosine
  modified_base (5)..(5): May be locked nucleic acid cytidine
  modified_base (7)..(7): May be locked nucleic acid adenosine
  modified_base (9)..(9): May be locked nucleic acid thymidine
  modified_base (10)..(10): May be locked nucleic acid adenosine
  modified_base (12)..(12): May be locked nucleic acid cytidine
  modified_base (13)..(13): May be locked nucleic acid adenosine
  modified_base (15)..(15): May be locked nucleic acid thymidine
  modified_base (16)..(16): May be locked nucleic acid adenosine
atcacgattagcatta

SEQ ID NO: 10 - DNA, 16 bp, Artificial Sequence
  oligonucleotide inhibitor of miR-155-5p
  misc_feature (1)..(16): May be joined through phosphorothioate bonds
  modified_base (1)..(1): May be locked nucleic acid adenosine
  modified_base (4)..(4): May be locked nucleic acid adenosine
  modified_base (7)..(7): May be locked nucleic acid adenosine
  modified_base (8)..(8): May be locked nucleic acid thymidine
  modified_base (10)..(10): May be locked nucleic acid adenosine
  modified_base (11)..(11): May be locked nucleic acid guanosine
  modified_base (13)..(13): May be locked nucleic acid adenosine
  modified_base (15)..(15): May be locked nucleic acid thymidine
  modified_base (16)..(16): May be locked nucleic acid adenosine
atcacgattagcatta

SEQ ID NO: 11 - DNA, 16 bp, Artificial Sequence
  oligonucleotide inhibitor of miR-155-5p
  misc_feature (1)..(16): May be joined through phosphorothioate bonds
  modified_base (1)..(1): May be locked nucleic acid adenosine
  modified_base (3)..(3): May be locked nucleic acid cytidine
  modified_base (6)..(6): May be locked nucleic acid guanosine
  modified_base (8)..(9): May be locked nucleic acid thymidine
  modified_base (12)..(12): May be locked nucleic acid cytidine
  modified_base (13)..(13): May be locked nucleic acid adenosine
  modified_base (15)..(15): May be locked nucleic acid thymidine
  modified_base (16)..(16): May be locked nucleic acid adenosine
atcacgattagcatta

SEQ ID NO: 12 - DNA, 16 bp, Artificial Sequence
  oligonucleotide inhibitor of miR-155-5p
  misc_feature (1)..(16): May be joined through phosphorothioate bonds
  modified_base (1)..(1): May be locked nucleic acid adenosine
  modified_base (2)..(2): May be locked nucleic acid thymidine
  modified_base (4)..(4): May be locked nucleic acid adenosine
  modified_base (5)..(5): May be locked nucleic acid cytidine
  modified_base (9)..(9): May be locked nucleic acid thymidine
  modified_base (10)..(10): May be locked nucleic acid adenosine
  modified_base (12)..(12): May be locked nucleic acid cytidine
  modified_base (13)..(13): May be locked nucleic acid adenosine
  modified_base (16)..(16): May be locked nucleic acid adenosine
atcacgattagcatta

SEQ ID NO: 13 - DNA, 16 bp, Artificial Sequence
  oligonucleotide inhibitor of miR-155-5p
  misc_feature (1)..(16): May be joined through phosphorothioate bonds
  modified_base (1)..(1): May be locked nucleic acid adenosine
  modified_base (3)..(3): May be locked nucleic acid cytidine
  modified_base (7)..(7): May be locked nucleic acid adenosine
  modified_base (9)..(9): May be locked nucleic acid thymidine
  modified_base (10)..(10): May be locked nucleic acid adenosine
  modified_base (12)..(12): May be locked nucleic acid cytidine
  modified_base (13)..(13): May be locked nucleic acid adenosine
  modified_base (15)..(15): May be locked nucleic acid thymidine
  modified_base (16)..(16): May be locked nucleic acid adenosine
atcacgattagcatta

SEQ ID NO: 14 - DNA, 16 bp, Artificial Sequence
  oligonucleotide inhibitor of miR-155-5p
  misc_feature (1)..(16): May be joined through phosphorothioate bonds
  modified_base (1)..(1): May be locked nucleic acid adenosine
  modified_base (3)..(3): May be locked nucleic acid cytidine
  modified_base (5)..(5): May be locked nucleic acid cytidine
  modified_base (7)..(7): May be locked nucleic acid adenosine
  modified_base (9)..(9): May be locked nucleic acid thymidine
  modified_base (11)..(11): May be locked nucleic acid guanosine
  modified_base (13)..(13): May be locked nucleic acid adenosine
  modified_base (15)..(15): May be locked nucleic acid thymidine
  modified_base (16)..(16): May be locked nucleic acid adenosine
atcacgattagcatta

SEQ ID NO: 15 - DNA, 15 bp, Artificial Sequence
  oligonucleotide inhibitor of miR-155-5p
  misc_feature (1)..(15): May be joined through phosphorothioate bonds
  modified_base (1)..(1): May be locked nucleic acid thymidine
  modified_base (4)..(4): May be locked nucleic acid cytidine
  modified_base (7)..(7): May be locked nucleic acid thymidine
  modified_base (10)..(10): May be locked nucleic acid guanosine
  modified_base (12)..(12): May be locked nucleic acid adenosine
  modified_base (13)..(13): May be locked nucleic acid thymidine
  modified_base (15)..(15): May be locked nucleic acid adenosine
tcacgattagcatta

SEQ ID NO: 16 - DNA, 15 bp, Artificial Sequence
  oligonucleotide inhibitor of miR-155-5p
  misc_feature (1)..(15): May be joined through phosphorothioate bonds
  modified_base (1)..(1): May be locked nucleic acid thymidine
  modified_base (3)..(3): May be locked nucleic acid adenosine
  modified_base (6)..(6): May be locked nucleic acid adenosine
  modified_base (7)..(7): May be locked nucleic acid thymidine
  modified_base (10)..(10): May be locked nucleic acid guanosine
  modified_base (12)..(12): May be locked nucleic acid adenosine
  modified_base (14)..(14): May be locked nucleic acid thymidine
  modified_base (15)..(15): May be locked nucleic acid adenosine
tcacgattagcatta

SEQ ID NO: 17 - DNA, 15 bp, Artificial Sequence
  oligonucleotide inhibitor of miR-155-5p
  misc_feature (1)..(15): May be joined through phosphorothioate bonds
  modified_base (1)..(1): May be locked nucleic acid thymidine
  modified_base (5)..(5): May be locked nucleic acid guanosine
  modified_base (6)..(6): May be locked nucleic acid adenosine
  modified_base (7)..(7): May be locked nucleic acid thymidine
  modified_base (10)..(10): May be locked nucleic acid guanosine
  modified_base (12)..(12): May be locked nucleic acid adenosine
  modified_base (14)..(14): May be locked nucleic acid thymidine
  modified_base (15)..(15): May be locked nucleic acid adenosine
tcacgattagcatta

SEQ ID NO: 18 - DNA, 15 bp, Artificial Sequence
  oligonucleotide inhibitor of miR-155-5p
  misc_feature (1)..(15): May be joined through phosphorothioate bonds
  modified_base (1)..(1): May be locked nucleic acid thymidine
  modified_base (2)..(2): May be locked nucleic acid cytidine
  modified_base (3)..(3): May be locked nucleic acid adenosine
  modified_base (5)..(5): May be locked nucleic acid guanosine
  modified_base (8)..(8): May be locked nucleic acid thymidine
  modified_base (9)..(9): May be locked nucleic acid adenosine
  modified_base (11)..(11): May be locked nucleic acid cytidine
  modified_base (14)..(14): May be locked nucleic acid thymidine
  modified_base (15)..(15): May be locked nucleic acid adenosine
tcacgattagcatta

SEQ ID NO: 19 - DNA, 15 bp, Artificial Sequence
  oligonucleotide inhibitor of miR-155-5p
  misc_feature (1)..(15): May be joined through phosphorothioate bonds
  modified_base (1)..(1): May be locked nucleic acid thymidine
  modified_base (4)..(4): May be locked nucleic acid cytidine
  modified_base (8)..(8): May be locked nucleic acid thymidine
  modified_base (9)..(9): May be locked nucleic acid adenosine
  modified_base (10)..(10): May be locked nucleic acid guanosine
  modified_base (11)..(11): May be locked nucleic acid cytidine
  modified_base (12)..(12): May be locked nucleic acid adenosine
  modified_base (13)..(14): May be locked nucleic acid thymidine
  modified_base (15)..(15): May be locked nucleic acid adenosine
tcacgattagcatta

SEQ ID NO: 20 - DNA, 15 bp, Artificial Sequence
  oligonucleotide inhibitor of miR-155-5p
  misc_feature (1)..(15): May be joined through phosphorothioate bonds
  modified_base (1)..(1): May be locked nucleic acid thymidine
  modified_base (3)..(3): May be locked nucleic acid adenosine
  modified_base (5)..(5): May be locked nucleic acid guanosine
  modified_base (6)..(6): May be locked nucleic acid adenosine
  modified_base (7)..(7): May be locked nucleic acid thymidine
  modified_base (10)..(10): May be locked nucleic acid guanosine
  modified_base (11)..(11): May be locked nucleic acid cytidine
  modified_base (12)..(12): May be locked nucleic acid adenosine
  modified_base (14)..(14): May be locked nucleic acid thymidine
  modified_base (15)..(15): May be locked nucleic acid adenosine
tcacgattagcatta

SEQ ID NO: 21 - DNA, 11 bp, Artificial Sequence
  oligonucleotide inhibitor of miR-155-5p
  misc_feature (1)..(11): May be joined through phosphorothioate bonds
  modified_base (1)..(1): May be locked nucleic acid guanosine
  modified_base (2)..(2): May be locked nucleic acid adenosine
  modified_base (3)..(4): May be locked nucleic acid thymidine
  modified_base (5)..(5): May be locked nucleic acid adenosine
  modified_base (6)..(6): May be locked nucleic acid guanosine
  modified_base (8)..(8): May be locked nucleic acid adenosine
  modified_base (9)..(9): May be locked nucleic acid thymidine
  modified_base (11)..(11): May be locked nucleic acid adenosine
gattagcatta

SEQ ID NO: 22 - DNA, 12 bp, Artificial Sequence
  oligonucleotide inhibitor of miR-155-5p
  misc_feature (1)..(12): May be joined through phosphorothioate bonds
  modified_base (1)..(1): May be locked nucleic acid cytidine
  modified_base (3)..(3): May be locked nucleic acid adenosine
  modified_base (4)..(5): May be locked nucleic acid thymidine
  modified_base (6)..(6): May be locked nucleic acid adenosine
  modified_base (7)..(7): May be locked nucleic acid guanosine
  modified_base (9)..(9): May be locked nucleic acid adenosine
  modified_base (10)..(11): May be locked nucleic acid thymidine
  modified_base (12)..(12): May be locked nucleic acid adenosine
cgattagcatta

SEQ ID NO: 23 - DNA, 12 bp, Artificial Sequence
  oligonucleotide inhibitor of miR-155-5p
  misc_feature (1)..(12): May be joined through phosphorothioate bonds
  modified_base (1)..(1): May be locked nucleic acid cytidine
  modified_base (3)..(3): May be locked nucleic acid adenosine
  modified_base (4)..(5): May be locked nucleic acid thymidine
  modified_base (7)..(7): May be locked nucleic acid guanosine
  modified_base (9)..(9): May be locked nucleic acid adenosine
  modified_base (10)..(11): May be locked nucleic acid thymidine
  modified_base (12)..(12): May be locked nucleic acid adenosine
cgattagcatta

SEQ ID NO: 24 - DNA, 14 bp, Artificial Sequence
  oligonucleotide inhibitor of miR-155-5p
  misc_feature (1)..(14): May be joined through phosphorothioate bonds
  modified_base (1)..(1): May be locked nucleic acid cytidine
  modified_base (2)..(2): May be locked nucleic acid adenosine
  modified_base (4)..(4): May be locked nucleic acid guanosine
  modified_base (6)..(7): May be locked nucleic acid thymidine
  modified_base (9)..(9): May be locked nucleic acid guanosine
  modified_base (11)..(11): May be locked nucleic acid adenosine
  modified_base (12)..(13): May be locked nucleic acid thymidine
  modified_base (14)..(14): May be locked nucleic acid adenosine
cacgattagcatta

SEQ ID NO: 25 - DNA, 14 bp, Artificial Sequence
  oligonucleotide inhibitor of miR-155-5p
  misc_feature (1)..(14): May be joined through phosphorothioate bonds
  modified_base (1)..(1): May be locked nucleic acid cytidine
  modified_base (3)..(3): May be locked nucleic acid cytidine
  modified_base (6)..(7): May be locked nucleic acid thymidine
  modified_base (9)..(9): May be locked nucleic acid guanosine
  modified_base (11)..(11): May be locked nucleic acid adenosine
  modified_base (12)..(13): May be locked nucleic acid thymidine
  modified_base (14)..(14): May be locked nucleic acid adenosine
cacgattagcatta

SEQ ID NO: 26 - DNA, 15 bp, Artificial Sequence
  oligonucleotide inhibitor of miR-155-5p
  misc_feature (1)..(15): May be joined through phosphorothioate bonds
  modified_base (1)..(1): May be locked nucleic acid thymidine
  modified_base (3)..(3): May be locked nucleic acid adenosine
  modified_base (4)..(4): May be 5-Methylcytosine
  modified_base (5)..(5): May be locked nucleic acid guanosine
  modified_base (6)..(6): May be locked nucleic acid adenosine
  modified_base (7)..(7): May be locked nucleic acid thymidine
  modified_base (10)..(10): May be locked nucleic acid guanosine
  modified_base (11)..(11): May be locked nucleic acid cytidine
  modified_base (12)..(12): May be locked nucleic acid adenosine
  modified_base (14)..(14): May be locked nucleic acid thymidine
  modified_base (15)..(15): May be locked nucleic acid adenosine
tcacgattagcatta

SEQ ID NO: 27 - DNA, 8 bp, Artificial Sequence
  oligonucleotide inhibitor of miR-155-5p
  misc_feature (1)..(8): May be joined through phosphorothioate bonds
  modified_base (1)..(1): May be locked nucleic acid thymidine
  modified_base (2)..(2): May be locked nucleic acid adenosine
  modified_base (3)..(3): May be locked nucleic acid guanosine
  modified_base (4)..(4): May be locked nucleic acid cytidine
  modified_base (5)..(5): May be locked nucleic acid adenosine
  modified_base (6)..(7): May be locked nucleic acid thymidine
  modified_base (8)..(8): May be locked nucleic acid adenosine
tagcatta

SEQ ID NO: 28 - RNA, 23 nt, Mus sp.
uuaaugcuaauugugauaggggu

(19) United States
(12) Patent Application Publication
(10) Pub. No.: US 2018/0161357 A1
(43) Pub. Date: Jun. 14, 2018

(54) MIR-155 INHIBITORS FOR TREATING AMYOTROPHIC LATERAL SCLEROSIS (ALS)

(71) Applicants: MIRAGEN THERAPEUTICS, INC., Boulder, CO (US); The Brigham and Women's Hospital, Inc., Boston, MA (US)

(72) Inventors: Aimee JACKSON, Boulder, CO (US); Christina DALBY, Boulder, CO (US); Howard L. WEINER, Brookline, MA (US); Oleg BUTOVSKY, Needham, MA (US)

(21) Appl. No.: 15/579,718

(22) PCT Filed: Jun. 3, 2016
(86) PCT No.: PCT/US2016/035794

§ 371 Date: Dec. 5, 2017

Related U.S. Application Data
(60) Provisional application No. 62/171,743, filed on Jun. 5, 2015.

(51) Int. Cl. A61K 31/712; A61K 31/7125; A61P 25/28

(52) U.S. Cl. CPC A61K 31/712; A61P 25/28; A61K 31/7125

(57) ABSTRACT

The present invention provides methods for treating a neurological disease such as ALS in a subject by administering to the subject an oligonucleotide inhibitor of miR-155. The invention also provides methods for treating neuroinflammation by administering an oligonucleotide inhibitor of miR-155.

### CROSS REFERENCE TO RELATED APPLICATIONS

The present Application claims the benefit of priority to U.S. Provisional Application No. 62/171,743, filed on Jun. 5, 2015, the contents of which are hereby incorporated by reference in their entirety.

### FIELD OF THE INVENTION

The present invention relates to oligonucleotide inhibitors of miR-155 and compositions thereof. The invention also provides methods for treating or preventing a neurological disease and/or neuroinflammation in a subject in need thereof by administering an oligonucleotide inhibitor of miR-155. The activity or function of miR-155 is reduced in central nervous system (CNS) cells of the subject following administration of the oligonucleotide inhibitor.

### BACKGROUND

MicroRNA (miRNA) profiling of clinical samples has demonstrated that miR-155 is up-regulated in spinal cord and peripheral monocytes of both sporadic and familial amyotrophic lateral sclerosis (ALS) patients (Butovsky et al., 2012; Koval et al., 2013). ALS is a complex disease that may be initiated by a variety of neuropathic cellular mechanisms. Regardless of the initiating event, ALS is associated with local and systemic M1 polarization of monocytic inflammatory cells. In ALS patients as well as animal models, inflammation of non-neuronal cells including microglia contributes to neuronal death (Boillee et al., 2006; Nagai et al. 2007). M1 polarization of spinal cord microglial cells and circulating monocytes that traffic into the spinal cord is associated with, and at least partially the result of, increased expression of miR-155 (Butovsky et al., 2012). In a preclinical model of ALS, the SOD1G93A mouse, miR-155 expression is up-regulated in resident microglia and peripheral monocytes (Butovsky et al., 2012; Koval et al., 2013). Genetic deletion of miR-155 in the SOD1G93A mouse produces significantly prolonged survival (51 days), reduced recruitment of peripheral monocytes into the spinal cord, and reversal of the dysregulated microglial signature characteristic of the pathologic state in the SOD1G93A mouse (Butovsky et al., 2014). In addition, genetic ablation of miR-155 shifts the miRNA/gene and protein signature in splenic Ly6CHi monocytes and spinal cord of SOD1G93A mice from the tissue destructive M1 type to the tissue protective M2 type. Furthermore, pharmacological inhibition of miR-155 by direct (intracerebroventricular injection) or systemic administration also improves survival of SOD1G93A mice (Koval et al., 2013; Butovsky et al., 2014) and reverses the disease-associated microglial signature (Butovsky et al., 2014).

Prior studies evaluating pharmacological administration of antimiR-155 (Koval et al., 2013; Butovsky et al., 2014) utilized compounds targeting the mouse miR-155. The mouse miR-155 sequence (UUAAUGCUAAUUGUGAUAGGGGU) differs from the human miR-155 sequence (UUAAUGCUAAUCGUGAUAGGGGU) by a single nucleotide at position 12 of the guide strand (miRBase 21, www.mirbase.org). Although previous studies have shown the role of antimiR-155 compounds in the treatment of ALS, the present invention provides additional oligonucleotide inhibitors that down-regulate the activity or function of human miR-155.

### SUMMARY OF THE INVENTION

The present invention provides oligonucleotide inhibitors for modulating the activity or function of miR-155 in cells of a subject. In one embodiment, administration of an oligonucleotide inhibitor of miR-155 down-regulates the activity or function of miR-155 in CNS cells of the subject following administration. In certain embodiments, the CNS cells are monocytes, lymphocytes, microglia, macrophages, and neuronal cells. In some embodiments, CNS cells include cells in peripheral blood that can migrate into the spinal cord; e.g. peripheral blood monocytes, peripheral blood lymphocytes, etc.

In one embodiment, the oligonucleotide inhibitor of miR-155 comprises a sequence of 11 to 16 nucleotides, wherein the oligonucleotide inhibitor is fully complementary to a mature sequence of miR-155 and has a full phosphorothioate backbone; and wherein at least the first three nucleotides from the 3′ end of the oligonucleotide inhibitor are locked nucleotides and at least the second nucleotide from the 5′ end of the oligonucleotide inhibitor is a deoxyribonucleic acid (DNA) nucleotide. In further embodiments, the fourth nucleotide from the 3′ end of the oligonucleotide inhibitor is a locked nucleotide and/or the sixth nucleotide from the 5′ end of the oligonucleotide inhibitor is a DNA nucleotide.

In one embodiment, the oligonucleotide inhibitor of miR-155 has a length of 12 nucleotides. In some embodiments, the oligonucleotide inhibitor contains at least 9 locked nucleotides. In some other embodiments, the oligonucleotide inhibitor contains up to 1, 2, 3, 4, or 5 DNA nucleotides. In certain embodiments, at least the second nucleotide from the 5′ end of the oligonucleotide inhibitor is a DNA nucleotide. In further embodiments, at least the sixth and/or the eighth nucleotide from the 5′ end of the oligonucleotide inhibitor is a DNA nucleotide. In yet further embodiments, the oligonucleotide inhibitor comprises DNA nucleotides at the second, sixth, and the eighth position from the 5′ end.

In one embodiment, oligonucleotide inhibitors of miR-155 according to the present invention reduce or inhibit the activity of inflammatory cells of the CNS. Inflammatory cells include lymphocytes, monocytes, macrophages and microglia. In one embodiment, cells in peripheral blood such as monocytes, lymphocytes, NK cells, neutrophils, etc. can migrate into the spinal cord and act as inflammatory cells of the CNS. In some embodiments, oligonucleotide inhibitors of the present invention down-regulate the recruitment or migration of inflammatory cells into the spinal cord.

In another embodiment, oligonucleotide inhibitors up-regulate one or more target genes of miR-155 in CNS cells. In yet another embodiment, oligonucleotide inhibitors of the present invention up-regulate the expression or activity of homeostatic genes in cells of the CNS. In yet another embodiment, oligonucleotide inhibitors of the present invention down-regulate the expression or activity of tissue-destructive genes and/or up-regulates the expression or activity of tissue-protective genes in cells of the CNS.

The present invention also provides compositions comprising oligonucleotide inhibitors of miR-155 and uses thereof. In one embodiment, the invention provides methods for treating a neurological disease in a subject in need thereof, comprising administering to the subject a therapeutically effective amount of an oligonucleotide inhibitor of miR-155 of the present invention. The activity or function of miR-155 is reduced in CNS cells of the subject following administration of the oligonucleotide inhibitor, in one embodiment, the neurological disease is ALS.

In one embodiment, the invention provides methods for treating or ameliorating neuro-inflammation in a subject in need thereof, comprising administering to the subject the oligonucleotide inhibitor of the present invention. In these embodiments, the subject in need of a treatment for neuroinflammation may be suffering from or is at the risk of developing a neurological disease such as ALS.

### BRIEF DESCRIPTION OF THE DRAWINGS

FIG. 1 shows the expression of two direct seed-matched target genes of miR-155 in MV4-11 human monocytic cells transfected with antimiR-155 compounds.

FIG. 2A shows the relative expression of a direct seed-matched target, CSF1R, in microglial cells passively incubated with antimiR-155 compounds compared to untreated cells. FIG. 2B shows the relative expression of a second seed-matched target, OLFML3, in microglial cells passively incubated with antimiR-155 compounds compared to untreated cells.

FIG. 3 shows a “heat map” representation of gene expression changes in predicted or validated seed-matched targets of miR-155 in microglial cells isolated from SOD1 mice treated with antimiR-155 compounds.

FIG. 4 shows a fold-change in the expression of a set of miR-155 target genes up-regulated in ≥4 mice by ≥2 antimiR-155 compounds.

FIG. 5 shows an annotated gene expression profile for microglial homeostatic genes in mice treated with antimiR-155 compounds.

FIG. 6 shows a fold-change in the expression of a set of microglial homeostatic genes up-regulated in ≥4 mice by ≥2 antimiR-155 compounds.

### DETAILED DESCRIPTION

The present invention provides oligonucleotide inhibitors that inhibit the activity or function of miR-155 and compositions and uses thereof. In humans, miR-155 is encoded by the MIR155 host gene or MIR155HG and is located on human chromosome 21. Since both arms of pre-miR-155 can give rise to mature miRNAs, processing products of pre-miR-155 are designated as miR-155-5p (from the 5′ arm) and miR-155-3p (from the 3′ arm). The mature sequences for human miR-155-5p and miR-155-3p are given below:

Human mature raiR-155-5p
(SEQ ID NO: 1)
5′-UUAAUGCUAAUCGUGAUAGGGGU-3′
Human mature miR-155-3p
(SEQ ID NO: 2)
5′-CUCCUACAUAUUAGCAUUAACA-3′

miR-155-5p is expressed in hematopoietic cells including B-cells, T-cells, monocytes and granulocytes (Landgraf et al. 2007), miR-155-5p is an essential molecule in the control of both myelopoiesis and erythropoiesis. This miRNA is highly expressed in hematopoietic stem-progenitor cells at an early stem-progenitor stage; and blocks their differentiation into a more mature hematopoietic cell e.g., lymphocyte, erythrocyte). miR-155-5p expression progressively decreases as cells mature along these lineages, and is ˜200-fold lower in mature hematopoietic cells (Masaki et al. 2007; Gerloff et al. 2015).

Previous studies indicate that miR-155 is up-regulated in spinal cords and peripheral monocytes of ALS patients. U.S. application Ser. No. 14/350,977 (published as US 2014/0235697) discloses methods of diagnosing and treating neurodegenerative diseases, e.g. ALS, by administering an inhibitor of miR-155. This application is hereby incorporated by reference in its entirety for all purposes.

The present invention provides oligonucleotide inhibitors that reduce or inhibit ti activity or function of human miR-155. In the context of the present invention, the term “oligonucleotide inhibitor”, “antimiR”, “antagonist”, “antisense oligonucleotide or ASO”, “oligomer”, “anti-microRNA oligonucleotide or AMO”, or “mixmer” is used broadly and encompasses an oligomer comprising ribonucleotides, deoxyribonucleotides, modified ribonucleotides, modified deoxyribonucleotides or a combination thereof, that inhibits the activity or function of the target microRNA (miRNA) by fully or partially hybridizing to the miRNA thereby repressing the function or activity of the target miRNA.

The term “miR-155” as used herein includes pri-miR-155, pre-miR-155, miR-155-5p, and hsa-miR-155-5p.

In one embodiment, the present invention provides an oligonucleotide inhibitor of miR-155 that has a length of 11 to 16 nucleotides. In various embodiments, the oligonucleotide inhibitor targeting miR-155 is 11, 12, 13, 14, 15, or 16 nucleotides in length. In one embodiment, the oligonucleotide inhibitor of miR-155 has a length of 12 nucleotides.

The sequence of an oligonucleotide inhibitor of miR-155 according to the invention is sufficiently complementary to a mature sequence of miR-155-5p to hybridize to miR-155-5p under physiological conditions and inhibit the activity or function of miR-155-5p in the cells of a subject. For instance, in some embodiments, oligonucleotide inhibitors comprise a sequence that is at least partially complementary to a mature sequence of miR-155-5p, e.g. at least about 75%, 80%, 85%, 90%, 95%, 96%, 97%, 98%, or 99% complementary to a mature sequence of miR-155-5p. In some embodiments, the oligonucleotide inhibitor can be substantially complementary to a mature sequence of miR-155-5p, that is at least about 90%, 95%, 96%, 97%, 98%, or 99% complementary to a mature sequence of miR-155-5p. In one embodiment, the oligonucleotide inhibitor comprises a sequence that is 100% or fully complementary to a mature sequence of miR-155-5p. It is understood that the sequence of the oligonucleotide inhibitor is considered to be complementary to miR-155 even if the oligonucleotide, inhibitor sequence includes a modified nucleotide instead of a naturally-occurring nucleotide. For example, if a mature sequence of miR-155 comprises a guanosine nucleotide at a specific position, the oligonucleotide inhibitor may comprise a modified cytidine nucleotide, such as a locked cytidine nucleotide or 2′-fluoro-cytidine, at the corresponding position.

The term “about” as used herein is meant to encompass variations of +/−10% and more preferably +/−5%, as such variations are appropriate for practicing the present invention.

In some embodiments, the entire sequence of the oligonucleotide inhibitor of miR-155 is fully complementary to a mature sequence of human miR-155-5p. In various embodiments, the mature sequence of human miR-155-5p to which the sequence of the oligonucleotide inhibitor of the present invention is partially, substantially, or fully complementary to includes nucleotides 1-17, or nucleotides 2-17, or nucleotides 2-16, or nucleotides 2-15, or nucleotides 2-14, or nucleotides 2-13, or nucleotides 2-12 from the 5′ end of SEQ ID NO: 1. In one embodiment, the mature sequence of human miR-155-5p to which the sequence of the oligonucleotide inhibitor of the present invention is partially, substantially, or fully complementary to includes nucleotides 2-13 from the 5′ end of SEQ NO: 1.

In one embodiment, the oligonucleotide inhibitor of miR-155 contains at least one backbone modification, such as at least one phosphorothioate, morpholino, or phosphonocarboxylate internucleotide linkage (see, for example, U.S. Pat. Nos. 6,693,187 and 7,067,641, which are herein incorporated by reference in their entireties). In certain embodiments, the oligonucleotide inhibitor of miR-155 is fully phosphorothioate-linked.

In one embodiment, the oligonucleotide inhibitor of miR-155 contains at least one modified nucleotide such as a locked nucleotide or a nucleotide containing other sugar or base modifications. The terms “locked nucleotide.” “locked nucleic acid unit,” “locked nucleic acid residue,” or “LNA unit” may be used interchangeably throughout the disclosure and refer to a bicyclic nucleoside analogue. For instance, suitable oligonucleotide inhibitors can be comprised of one or more “conformationally constrained” or bicyclic sugar nucleoside modifications (BSN) that confer enhanced thermal stability to complexes formed between the oligonucleotide containing BSN and their complementary target strand. In one embodiment, the oligonucleotide inhibitors contain locked nucleotides or LNAs containing the 2′-O, 4′-C-methylene ribonucleoside (structure A) wherein the ribose sugar moiety is in a “locked” conformation. In another embodiment, the oligonucleotide inhibitors contain at least one 2′,4′-C-bridged 2′ deoxyribonucleoside (CDNA, structure B). See. e.g., U.S. Pat. No. 6,403,566 and Wang et al. (1999) Bioorganic and Medicinal Chemistry Letters, Vol. 9: 1147-1150, both of which are herein incorporated by reference in their entireties. In yet another embodiment, the oligonucleotide inhibitors contain at least one modified nucleoside having the structure shown in structure C. The oligonucleotide inhibitors targeting miR-155 can contain combinations of BSN (LNA, CDNA and the like) or other modified nucleotides, and ribonucleotides or deoxyribonucleotides.

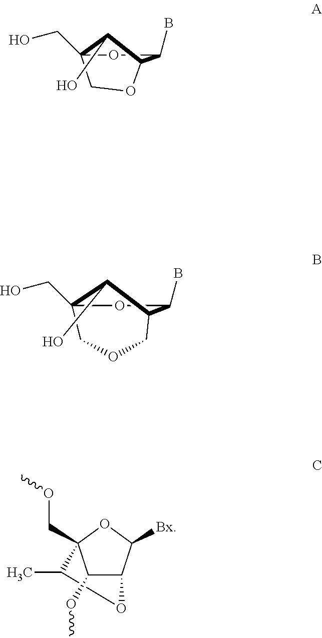

When referring to substituting a DNA or RNA nucleotide by its corresponding locked nucleotide in the context of the present invention, the term “corresponding locked nucleotide” is intended to mean that the DNA/RNA nucleotide has been replaced by a locked nucleotide containing the same naturally-occurring nitrogenous base as the DNA/RNA nucleotide that it has replaced or the same nitrogenous base that is chemically modified. For example, the corresponding locked nucleotide of a DNA nucleotide containing the nitrogenous base C may contain the same nitrogenous base C or the same nitrogenous base C that is chemically modified, such as 5-methylcytosine.

The term “non-locked nucleotide” refers to a nucleotide different from a locked-nucleotide, i.e. the term “non-locked nucleotide” includes a DNA nucleotide, an RNA nucleotide as well as a modified nucleotide where a base and/or sugar is modified except that the modification is not a locked modification.

In one embodiment, the oligonucleotide inhibitor of miR-155 contains at least 9 locked nucleotides. In one embodiment, at least the first three nucleotides from the 3′ end of the oligonucleotide inhibitor are locked nucleotides. In another embodiment, at least the first four nucleotides from the 3′ end of the oligonucleotide inhibitor are locked nucleotides. In yet another embodiment, the first nucleotide from the 5′ end of the oligonucleotide inhibitor is a locked nucleotide.

In certain embodiments, the oligonucleotide inhibitor contains at least 1, at least 2, at least 3, at least 4, or at least 5 DNA nucleotides. In one embodiment, at least the second nucleotide from the 5′ end of the oligonucleotide inhibitor is a DNA nucleotide. In another embodiment, at least the second and the sixth nucleotides from the 5′ end of the oligonucleotide inhibitor are DNA nucleotides. In yet another embodiment, at least the second, sixth and the eighth nucleotides from the 5′ end of the oligonucleotide inhibitor are DNA nucleotides.

In one embodiment, the oligonucleotide inhibitor of miR-155 comprises a sequence of 11 to 16 nucleotides, wherein the oligonucleotide inhibitor is fully complementary to a mature sequence of miR-155 and has a full phosphorothioate backbone; and wherein at least the first three nucleotides from the 3′ end of the oligonucleotide inhibitor are locked nucleotides and at least the second nucleotide from the 5′ end of the oligonucleotide inhibitor is a deoxyribonucleic acid (DNA) nucleotide. In a further embodiment, the fourth nucleotide from the 3′ end of the oligonucleotide inhibitor is also a locked nucleotide. In a yet further embodiment, the sixth nucleotide from the 5′ end of the oligonucleotide inhibitor is a DNA nucleotide. In certain embodiments, the oligonucleotide inhibitor of miR-155 has a length of 12 nucleotides. In some embodiments, the oligonucleotide inhibitor contains at least 9 locked nucleotides.

In one embodiment, the oligonucleotide inhibitor of miR-155 comprises a sequence of SEQ ID NO: 23.

In various embodiments, the oligonucleotide inhibitor of miR-155-5p has a sequence selected from Table 1.

TABLE 1

| SEQ ID NO. | Sequence (5′-3′) with modifications¹ |
|---|---|
| SEQ ID NO: 3 | 5′-lAs.dTs.dCs.dAs.lCs.lGs.dAs.lTs.dTs.lAs.lGs.dCs.lAs.dTs.lTs.lA-3′ |
| SEQ ID NO: 4 | 5′-lAs.dTs.dCs.dAs.lCs.lGs.dAs.dTs.lTs.lAs.lGs.dCs.lAs.dTs.lTs.lA-3′ |
| SEQ ID NO: 5 | 5′-lAs.lTs.dCs.dAs.dCs.lGs.dAs.lfs.dTs.lAs.lGs.dCs.lAs.dTs.lTs.lA-3′ |
| SEQ ID NO: 6 | 5′-lAs.lTs.dCs.dAs.dCs.lGs.lAs.dTs.dTs.lAs.lGs.lCs.dAs.lTs.dTs.lA-3′ |
| SEQ ID NO: 7 | 5′-lAs.dTs.dCs.dAs.lCs.lGs.dAs.lTs.dTs.lAs.lGs.dCs.lAs.lTs.dTs.lA-3′ |
| SEQ ID NO: 8 | 5′-lAs.lTs.dCs.dAs.lCs.dGs.dAs.dTs.lTs.lAs.dGs.lCs.lAs.dTs.lTs.lA-3′ |
| SEQ ID NO: 9 | 5′-lAs.dTs.dCs.dAs.lCs.dGs.lAs.dTs.lTs.lAs.dGs.lCs.lAs.dTs.lTs.lA-3′ |
| SEQ ID NO: 10 | 5′-lAs.dTs.dCs.lAs.dCs.dGs.lAs.lTs.dTs.lAs.lGs.dCs.lAs.dTs.lTs.lA-3′ |
| SEQ ID NO: 11 | 5′-lAs.dTs.lCs.dAs.dCs.lGs.dAs.lTs.lTs.dAs.dGs.lCs.lAs.dTs.lTs.lA-3′ |
| SEQ ID NO: 12 | 5′-lAs.lTs.dCs.lAs.lCs.dGs.dAs.dTs.lTs.lAs.dGs.lCs.lAs.dTs.dTs.lA-3′ |
| SEQ ID NO: 13 | 5′-lAs.dTs.lCs.dAs.dCs.dGs.lAs.dTs.lTs.lAs.dGs.lCs.lAs.dTs.lTs.lA-3′ |
| SEQ ID NO: 14 | 5′-lAs.dTs.lCs.dAs.lCs.dGs.lAs.dTs.lTs.dAs.lGs.dCs.lAs.dTs.lTs.lA-3′ |
| SEQ ID NO: 15 | 5′-lTs.dCs.dAs.lCs.dGs.dAs.lTs.dTs.dAs.lGs.dCs.lAs.lTs.dTs.lA-3′ |
| SEQ ID NO: 16 | 5′-lTs.dCs.lAs.dCs.dGs.lAs.lTs.dTs.dAs.lGs.dCs.lAs.dTs.lTs.lA-3′ |
| SEQ ID NO: 17 | 5′-lTs.dCs.dAs.dCs.lGs.lAs.lTs.dTs.dAs.lGs.dCs.lAs.dTs.lTs.lA-3′ |
| SEQ ID NO: 18 | 5′-lTs.lCs.lAs.dCs.lGs.dAs.dTs.lTs.lAs.dGs.lCs.dAs.dTs.lTs.lA-3′ |
| SEQ ID NO: 19 | 5′-lTs.dCs.dAs.lCs.dGs.dAs.dTs.lTs.lAs.lGs.lCs.lAs.lTs.lTs.lA-3′ |
| SEQ ID NO: 20 | 5′-lTs.dCs:lAs.dCs.lGs.lAs.lTs.dTs.dAs.lGs.lCs.lAs.dTs.lTs.lA-3′ |
| SEQ ID NO: 21 | 5′-lGs.lAs.lTs.lTs.lAs.lGs.dCs.lAs.lTs.dTs.lA-3′ |
| SEQ ID NO: 22 | 5′-lCs:dGs.lAs.lTs.lTs.lAs.lGs.dCs.lAs.lTs.lTs.lA-3′ |
| SEQ ID NO: 23 | 5′-lCs.das.lAs.lTs.lTs.dAs.lGs.dCs.lAs.lTs.lTs.lA-3′ |
| SEQ ID NO: 24 | 5′-lCs.lAs.dCs.lGs.dAs.lTs.lTs.dAs.lGs.dCs.lAs.lTs.lTs.lA-3′ |
| SEQ ID NO: 25 | 5′-lCs.dAs.lCs.dGs.dAs.lTs.lTs.dAs.lGs.dCs.lAs.lT.lTs.lA-3′ |
| SEQ ID NO: 26 | 5′-lTs.dCs.lAs.mdCs.lGs.lAs.lTs.dTs.dAs.lGs.lCs.lAs.dTs.lTs.lA-3′ |
| SEQ ID NO: 27 | 5′-lTs.lAs.lGs.lCs.lAs.lTs.lTs.lA-3′ |

¹l = locked nucleic acid modification; d = deoxytibonucleotide; s = phosphorothioate linkage; md = 5-Methylcytosine.

Oligonucleotide inhibitors of the present invention may include modified nucleotides that have a base modification or substitution. The natural or unmodified bases in RNA are the purine bases adenine (A) and guanine (G), and the pyrimidine bases cytosine (C) and uracil (U) (DNA has thymine (T)). Modified bases, also referred to as heterocyclic base moieties, include other synthetic and natural nucleobases such as 5-methylcytosine (5-me-C), 5-hydroxymethyl cytosine, xanthine, hypoxanthine, 2-aminoadenine, 6-methyl and other alkyl derivatives of adenine and guanine, 2-propyl and other alkyl derivatives of adenine and guanine, 2-thiouracil, 2-thiothymine and 2-thiocytosine, 5-halouracil and cytosine, 5-propynyl uracil and cytosine and other alkynyl derivatives of pyrimidine bases, 6-azo cytosine and thymine, 5-uracil (pseudouracil), 4-thiouracil, 8-halo, 8-amino, 8-thiol, 8-thioalkyl, 8-hydroxyl and other 8-substituted adenines and guanines, 5-halo (including 5-bromo, 5-trifluoromethyl and other 5-substituted uracils and cytosines), 7-methylguanine and 7-methyladenine, 2-F-adenine, 2-amino-adenine, 8-azaguanine and 8-azaadenine, 7-deazaguanine and 7-deazaadenine and 3-deazaguanine and 3-deazaadenine. In certain embodiments, oligonucleotide inhibitors targeting miR-155 comprise one or more BSN modifications in combination with a base modification (e.g. 5-methyl cytidine).

Oligonucleotide inhibitors of the present invention may include nucleotides with modified sugar moieties. Representative modified sugars include carbocyclic or acyclic sugars, sugars having substituent groups at one or more of their 2′, 3′ or 4′ positions and sugars having substituents in place of one or more hydrogen atoms of the sugar. In certain embodiments, the sugar is modified by having a substituent group at the 2′ position. In additional embodiments, the sugar is modified by having a substituent group at the 3′ position. In other embodiments, the sugar is modified by having a substituent group at the 4′ position. It is also contemplated that a sugar may have a modification at more than one of those positions, or that an oligonucleotide inhibitor may have one or more nucleotides with a sugar modification at one position and also one or more nucleotides with a sugar modification at a different position.

Sugar modifications contemplated in the oligonucleotide inhibitors of the present invention include, but are not limited to, a substituent group selected from: OH; F; O-, S-, or N-alkyl; O-, S-, or N-alkenyl; O-, S- or N-alkynyl; or O-alkyl-O-alkyl, wherein the alkyl, alkenyl and alkynyl may be substituted or unsubstituted with C₁ to C₁₀ alkyl or C₂ to C₁₀ alkenyl and alkynyl. In one embodiment, the modification includes 2′-methoxyethoxy (2′-O—CH₂CH₂OCH₃, which is also known as 2′-O-(2-methoxyethyl) or 2′-MOE), that is, an alkoxyalkoxy group. Another modification includes 2′-dimethylaminooxyethoxy, that is, a O(CH₂)₂ON(CH₃)₂ group, also known as 2′-DMAOE and 2′-dimethylaminoethoxyethoxy (also known in the art as 2′-O-dimethyl-amino-ethoxy-ethyl or 2′-DMAEOE), that is, 2′-O—CH₂—O—CH₂—N(CH₃)₂.

Additional sugar substituent groups include allyl (—CH₂—CH═CH₂), —O-allyl, methoxy (—O—CH₃), aminopropoxy (—OCH₂CH₂CH₂NH₂), and fluoro (F). Sugar substituent groups on the 2′ position (2′−) may be in the arabino (up) position or ribo (down) position. One 2′-arabino modification is 2′-F. Other similar modifications may also be made at other positions on the sugar moiety, particularly the 3′ position of the sugar on the 3′ terminal nucleoside or in 2′-5′ linked oligonucleotides and the 5′ position of 5′ terminal nucleotide. In certain embodiments, the sugar modification is a 2′-O-alkyl (e.g. 2′-O-methyl, 2′-O-methoxyethyl), 2′-halo (e.g., 2′-fluoro, 2′-chloro, 2′-bromo), and 4′ thio modifications.

Other modifications of oligonucleotide inhibitors to enhance stability and improve efficacy, such as those described in U.S. Pat. No. 6,838,283, which is herein incorporated by reference in its entirety, are known in the art and are suitable for use in the methods of the invention. For instance, to facilitate in vivo delivery and stability, the oligonucleotide inhibitor can be linked to a steroid, such as cholesterol moiety, a vitamin, a fatty acid, a carbohydrate or glycoside, a peptide, or other small molecule ligand at its 3′ end.

Administration of an oligonucleotide inhibitor of the present invention to a subject reduces or inhibits the activity or function of miR-155 in cells of the subject. In one embodiment, the oligonucleotide inhibitor reduces the activity or function of miR-155 in cells of the central nervous system (CNS). The terms “cells of the CNS” or “inflammatory cells of the CNS” as used herein include lymphocytes, monocytes, macrophages, glial cells such as microglia and astrocytes, and neuronal cells. In one embodiment, the cells of the CNS are peripheral or circulating monocytes or peripheral blood lymphocytes that can migrate into the spinal cord. In another embodiment, the cells of the CNS are microglia.

In some embodiments, certain oligonucleotide inhibitors of the present invention may show a greater inhibition of the activity or function of miR-155 in cells of the CNS, such as peripheral monocytes or microglia, compared to other miR-155 inhibitors. The term “other miR-155 inhibitors” includes nucleic acid inhibitors such as antisense oligonucleotides, antimiRs, antagomiRs, mixmers, gaptners, aptamers, ribozymes, small interfering RNAs, or small hairpin RNAs; antibodies or antigen binding fragments thereof; and/or drugs, which inhibit the expression or activity of miR-155. It is possible that a particular oligonucleotide inhibitor of the present invention may show a greater inhibition of miR-155 in CNS cells compared to other oligonucleotide inhibitors of the present invention. The term “greater” as used herein refers to quantitatively more or statistically significantly more.

Administration of an oligonucleotide inhibitor of the present invention up-regulates the expression or activity of miR-155 target genes in cells of the subject. Target genes for miR-155 include, but are not limited to, IL7r, Tlr6, Mef2a, Inpp5d, Cttnbp2n1, 1810011O10Rik, Fads1, Cux1, Ap3d1, X99384, Olfm13, Mafb, Csf1r, Tgfbr2, Bach1, Sall1, Rapgef5, CEBPB, CCnd1, Msr1, Jarid2, Mr1, Gnas, and Mecp2. In one embodiment, oligonucleotide inhibitors of the present invention up-regulate the expression or activity of at least four target genes of miR-155 in cells of the CNS. In some embodiments, target genes up-regulated by oligonucleotides of the present invention include IL7r, Tlr6, Mef2a, Inpp5d, Cttnbp2n1, Sall1, Jarid2, Mr1, Gnas, and Mecp2. In another embodiment, oligonucleotide inhibitors of the present invention up-regulate the expression or activity of homeostatic genes in cells of the CNS. The invention encompasses using the changes in the expression of four or more genes (gene expression signature) or changes in the expression of homeostatic genes as means to determine the activity of miR-155 inhibitors. In some embodiments, there is about 1.5 fold, 2-fold, 3-fold, 4-fold, 5-fold, 6-fold, 7-fold, or 8-fold, including values therebetween, change in the expression or activity of miR-155 target genes upon administration of oligonucleotide inhibitors of the present invention. In one embodiment, there is at least about 2-fold, 3-fold, 4-fold, or 5-fold, including values therebetween, change in the expression or activity of miR-155 target genes upon administration of oligonucleotide inhibitors of the present invention.

In one embodiment, the oligonucleotide inhibitor of the present invention shows a greater up-regulation of miR-155 target genes in CNS cells compared to other miR-155 inhibitors. In some embodiments, the oligonucleotide inhibitors of the present invention show a greater up-regulation of at least four target genes of miR-155 in cells of the CNS compared to other miR-155 inhibitors. In some other embodiments, the oligonucleotide inhibitors of the present invention show a greater up-regulation of homeostatic genes in cells of the CNS compared to other miR-155 inhibitors. In one embodiment, the oligonucleotide inhibitors of the present invention show a greater up-regulation of the expression or activity of one or more genes selected from the group consisting of IL7r, Tlr6, Mef2a, Inpp5d, Cttnbp2n1, Sall1, Jarid2, Mr1, Gnas, and Mecp2, in CNS cells compared to other miR-155 inhibitors. In another embodiment, the oligonucleotide inhibitors of the present invention show a greater up-regulation of the activity or function of homeostatic genes, in CNS cells compared to other miR-155 inhibitors. In various embodiments, “greater up-regulation” includes about 2-fold, 3-fold, 4-fold, or 5-fold, including values therebetween, increase in the expression or activity of miR-155 target genes compared to other miR-155 inhibitors.

In some embodiments, oligonucleotide inhibitors of the present invention reduce or inhibit the activity of inflammatory cells of the CNS. It has been shown that inflammation of non-neuronal cells including microglia contributes to neuronal death in ALS (Boillee et al., 2006; Nagai et al., 2007). The term “activity of inflammatory cells of the CNS” refers to one or more inflammatory responses mediated by monocytes and microglia of the CNS. Inflammatory responses include, but are not limited to, secretion of cytokines and/or chemokines, chemotaxis, migration or infiltration of cells of the immune system such as monocytes, macrophages, neutrophils to the inflamed area, phagocytosis, release of reactive oxygen species and nitric oxide, etc. In one embodiment, oligonucleotide inhibitors of the present invention down-regulate the activity of inflammatory cells of the CNS by down-regulating the inflammatory responses mediated by cells such as monocytes and microglia. For example, in one embodiment, oligonucleotide inhibitors down-regulate the migration or recruitment of circulating monocytes into the spinal cord of subjects suffering from neuroinflammation. In another embodiment, oligonucleotide inhibitors of the present invention down-regulate the production of inflammatory cytokines such as TNFα, IL-1β, IL-6 by monocytes, macrophages and/or microglial cells of the CNS.

It is known that depending on the local tissue environment, monocytes, macrophages, and microglia can differentiate/polarize into M1 (pro-inflammatory/tissue destructive) or M2 (anti-inflammatory/tissue protective) phenotype. In one embodiment, oligonucleotide inhibitors of the present invention up-regulate the expression or activity of genes in cells of the CNS that direct the polarization of monocytes, macrophages, and microglia towards M2/tissue protective phenotype.

The present inventions provides methods for treating a neurological disease in a subject in need thereof, comprising administering to the subject an oligonucleotide inhibitor of miR-155 according to the invention. The activity or function of miR-155 is reduced in cells of the CNS of the subject following administration of the oligonucleotide inhibitor. In one embodiment, the method for treating a neurological disease comprises administering an oligonucleotide inhibitor of miR-155 that has a sequence of 11 to 16 nucleotides, wherein the oligonucleotide inhibitor is fully complementary to a mature sequence of miR-155 and has a hall phosphorothioate backbone; and wherein at least the first three nucleotides from the 3′ end of said oligonucleotide inhibitor are locked nucleotides and at least the second nucleotide from the 5′ end of the oligonucleotide inhibitor is a deoxyribonucleic acid (DNA) nucleotide. In a further embodiment, the sixth nucleotide from the 5′ end of the oligonucleotide inhibitor is also a DNA nucleotide. Neurological diseases that can be treated according to the invention include amyotrophic lateral sclerosis (ALS), multiple sclerosis (MS), Alzheimer's disease (AD), Japanese Encephalitis Virus (JEV)—induced neuroinflammation, alcohol-induced neuroinflammation, acute and chronic central nervous system (CNS) injury including traumatic brain injury, autoimmune encephalomyelitis, Parkinson's disease (PD), Huntington's disease (HD), brain stroke, brain tumors, cardiac ischemia, age-related macular degeneration (AMD), retinitis pigmentosa (RP), and neuropathic pain. In certain embodiments, the method for treating a neurological disease comprises administering an oligonucleotide inhibitor having a sequence of SEQ ID NO: 23.

The invention also encompasses methods for treating or ameliorating neuroinflammation in a subject in need thereof by administering an oligonucleotide inhibitor of miR-155 according to the invention. The activity or function of miR-155 is reduced in cells of the CNS of the subject following administration of the oligonucleotide inhibitor. The subject in need of a treatment for neuroinflammation may be suffering from a neurological disease or is at the risk of developing a neurological disease such as ALS, multiple sclerosis, Alzheimer's disease, Japanese Encephalitis Virus—induced neuroinflammation, alcohol-induced neuroinflammation, acute and chronic central nervous system injury including traumatic brain injury, autoimmune encephalomyelitis, Parkinson's disease, Huntington's disease, brain stroke, brain tumors, cardiac ischemia, age-related macular degeneration, retinitis pigmentosa, and neuropathic pain.

In one embodiment, the invention provides methods for reducing or inhibiting the activity of inflammatory cells in a neurological disease, comprising administering the oligonucleotide inhibitor of the invention. The activity or function of miR-155 is reduced in inflammatory cells of the central nervous system (CNS) following administration of the oligonucleotide inhibitor. Administration of oligonucleotide inhibitors of the invention may down-regulate various activities of inflammatory cells such as secretion of cytokines and/or chemokines, chemotaxis, migration or infiltration of cells of the immune system such as monocytes, macrophages, neutrophils to the inflamed area, phagocytosis, release of reactive oxygen species and nitric oxide, dc. In one embodiment, oligonucleotide inhibitors reduce or inhibit the activity of inflammatory cells by down-regulating the recruitment or migration of inflammatory cells into the spinal cord. In another embodiment, oligonucleotide inhibitors reduce or inhibit the activity of inflammatory cells by down-regulating the expression of genes involved in M1/pro-inflammatory/tissue-destructive phenotype and/or up-regulating the genes involved in M2/anti-inflammatory/tissue-protective phenotype.

Preferably, administration of an oligonucleotide inhibitor of the present invention to the subject results in the improvement of one or more symptoms or pathologies associated with the neurological disease. For instance, in one embodiment, administration of an oligonucleotide inhibitor of the present invention to a patient suffering from ALS reduces muscle weakness in legs, hands, shoulders, arms and other body parts; reduces muscle cramps; improves speech; improves ability to walk, etc. In one embodiment, administration of an oligonucleotide inhibitor of the present invention reduces inflammation of neurons present in the CNS.

As used herein, the term “subject” or “patient” refers to any vertebrate including, without limitation, humans and other primates (e.g., chimpanzees and other apes and monkey species), farm animals (e.g., cattle, sheep, pigs, goats and horses), domestic mammals (e.g., dogs and cats), laboratory animals (e.g., rodents such as mice, rats, and guinea pigs), and birds (e.g., domestic, wild and game birds such as chickens, turkeys and other gallinaceous birds, ducks, geese, and the like). In some embodiments, the subject is a mammal. In other embodiments, the subject is a human.

Any of the oligonucleotide inhibitors of miR-155 described herein can be delivered to the target cell (e.g. monocytes) by delivering to the cell an expression vector encoding the miR-155 oligonucleotide inhibitor. A “vector” is a composition of matter which can be used to deliver a nucleic acid of interest to the interior of a cell. Numerous vectors are known in the art including, but not limited to, linear polynucleotides, polynucleotides associated with ionic or amphiphilic compounds, plasmids, and viruses. Thus, the term “vector” includes an autonomously replicating plasmid or a virus. Examples of viral vectors include, but are not limited to, adenoviral vectors, adeno-associated virus vectors, retroviral vectors, and the like. In one particular embodiment, the viral vector is a lentiviral vector or an adenoviral vector. An expression construct can be replicated in a living cell, or it can be made synthetically. For purposes of this application, the terms “expression construct,” “expression vector,” and “vector,” are used interchangeably to demonstrate the application of the invention in a general, illustrative sense, and are not intended to limit the invention.

In one embodiment, an expression vector for expressing an oligonucleotide inhibitor of miR-155 comprises a promoter operably linked to a polynucleotide sequence encoding the oligonucleotide inhibitor. The phrase “operably linked” or “under transcriptional control” as used herein means that the promoter is in the correct location and orientation in relation to a polynucleotide to control the initiation of transcription by RNA polymerase and expression of the polynucleotide.

As used herein, a “promoter” refers to a DNA sequence recognized by the synthetic machinery of the cell, or introduced synthetic machinery, required to initiate the specific transcription of a gene. Suitable promoters include, but are not limited to RNA pol I, pol II, pol III, and viral promoters (e.g. human cytomegalovirus (CMV) immediate early gene promoter, the SV40 early promoter, and the Rous sarcoma virus long terminal repeat). In one embodiment, the promoter is a monocyte specific promoter such as the CD14 promoter, CD68 promoter, etc. In another embodiment, the promoter is a microglia specific promoter such as the CX3CR1 promoter, the F4/80 promoter, etc.

In certain embodiments, the promoter operably linked to a polynucleotide encoding a miR-155 oligonucleotide inhibitor can be an inducible promoter. Inducible promoters are known in the art and include, but are not limited to, tetracycline promoter, metallothionein HA promoter, heat shock promoter, steroid/thyroid hormone/retinoic acid response elements, the adenovirus late promoter, and the inducible mouse mammary tumor virus LTR.

Methods of delivering expression constructs and nucleic acids to cells are known in the art and can include, for example, calcium phosphate co-precipitation, electroporation, microinjection, DEAE-dextran, lipofection, transfection employing polyamine transfection reagents, cell sonication, gene bombardment using high velocity microprojectiles, and receptor-mediated transfection.

The present invention also provides methods for diagnosing neurological diseases, e.g. ALS, and methods for monitoring clinical status of a patient undergoing the treatment for the neurological disease. For example, the invention shows that administration of antimiR-155 compounds of the invention up-regulates or down-regulates a unique set of genes in microglial cells isolated from SOD1 mouse (mouse model of ALS) compared to control-treated cells. The invention contemplates using a gene expression signature based on this unique set of genes to diagnose ALS as well to monitor progress of the ALS treatment with miR-155 inhibitors.

For instance, in one embodiment, the present invention provides methods for selecting a subject for treatment of ALS or neuroinflammation comprising determining a level of expression of one or more genes selected from the group consisting of IL7r, Tlr6, Mef2a, Inpp5d, Cttnbp2n1, 1810011O10Rik, Fads1, Cux1, Ap3d1, X99384, Olfm13, Mafb, Csf1r, Tgfbr2, Bach1, Sall1, Rapgef5, CEBPB, CCnd1, Msr1, Jarid2, Mr1, Gnas, and Mecp2 in CNS cells of the subject; comparing the level of the one or more genes in the CNS cells of the subject to a reference level of the same one or more genes; and selecting a subject having a decrease in the level of the one or more genes in the CNS cells compared to the reference level for treatment of ALS or neuroinflammation. In one embodiment, the method for selecting a subject for treatment of ALS or neuroinflammation comprises determining the level of at least 4 genes selected from the group consisting of, IL7r, Tlr6, Mef2a, Inpp5d, Cttnbp2n1, Sall1, Jarid2, Mr1 Gnas, and Mecp2, in CNS cells of the subject in comparison to a reference level of the same genes. In certain embodiments, the method for selecting a subject for treatment of ALS or neuroinflammation comprises determining the level of at least 4 genes selected from the group consisting of, IL7r, Tlr6, Mef2a, Inpp5d, Cttnbp2n1, Sall1, Jarid2, Mr1, Gnas, and Mecp2in CNS cells of the subject in comparison to a reference level of the same genes; and selecting a subject having at least 2-fold decrease in the level of the selected genes in the CNS cells compared to the reference level for treatment of ALS or neuroinflammation. In one embodiment, cells of the CNS may be obtained by obtaining cerebrospinal fluid (CSF) of the subject. In one embodiment, the reference level is the level of expression of the same genes in control oligonucleotide-treated cells. In another embodiment, the reference level is the level of expression of the same genes in from a healthy subject (e.g., a subject that does not present with two or more symptoms of a neurodegenerative disorder, a subject that has not been diagnosed with a neurodegenerative disorder, and/or a subject that has no family history of neurodegenerative disease).

The invention also provides methods for assessing the efficacy of a treatment with antimiR-155 compounds comprising determining a level of expression of one or more genes in cells of a subject prior to the treatment with antimiR-155 compounds, wherein the one or more genes are selected from a set of genes modulated in CNS cells, e.g. IL7r, Tlr6, Mef2a, Inpp5d, Cttnbp2n1, Sall1, Jarid2, Mr1, Gnas, and Mecp2; determining the level of expression of the same one or more genes in cells of the subject after treatment with antimiR-155 compounds; and determining the treatment to be effective, less effective, or not effective based on the expression levels prior to and after the treatment. That is, in one embodiment, target genes disclosed herein as up-regulated or down-regulated in response to antimiR-155 compounds serve as a biomarker for clinical efficacy of the antimiR-155 treatment.

The present invention also provides pharmaceutical compositions comprising an oligonucleotide inhibitor of miR-155 as disclosed herein and a pharmaceutically acceptable carrier or excipient. In one embodiment, the pharmaceutical composition comprises an effective dose of an oligonucleotide inhibitor of miR-155 having a sequence of 11 to 16 nucleotides, wherein the oligonucleotide inhibitor is fully complementary to a mature sequence of miR-155 and has a full phosphorothioate backbone; and wherein at least the first three nucleotides from the 3′ end of the oligonucleotide inhibitor are locked nucleotides and at least the second nucleotide from the 5′ end of the oligonucleotide inhibitor is a deoxyribonucleic acid (DNA) nucleotide. In certain embodiments, pharmaceutical compositions comprise an effective dose of an oligonucleotide inhibitor having a sequence of SEQ ID NO: 23. In yet other embodiments, the pharmaceutical composition comprises an oligonucleotide inhibitor having a sequence selected from the sequences listed in Table 1.

An “effective dose” is an amount sufficient to effect a beneficial or desired clinical result. An effective dose of an oligonucleotide inhibitor of miR-155 of the invention may be from about 1 mg/kg to about 100 mg/kg, about 2.5 mg/kg to about 50 ma/kg, or about 5 mg/kg to about 25 mg/kg. The precise determination of what would be considered an effective dose may be based on factors individual to each patient, including their size, age, type of disorder, and form of inhibitor (e.g. naked oligonucleotide or an expression construct etc.). Therefore, dosages can be readily ascertained by those of ordinary skill in the art from this disclosure and the knowledge in the art. Where clinical applications are contemplated, pharmaceutical compositions will be prepared in a form appropriate for the intended application. Generally, this will entail preparing compositions that are essentially free of pyrogens, as well as other impurities that could be harmful to humans or animals.

Pharmaceutical compositions of the present invention may be formulated for delivering oligonucleotide inhibitors systemically or locally (direct delivery) to the central nervous system. Delivery of pharmaceutical agents/compositions into the central nervous system (CNS) is challenging due to the presence of the blood-brain barrier. Various drug delivery strategies have been used to improve the delivery of an active agent across the blood-brain barrier (BBB) into the CNS. For example, to deliver a systemically administered active agent to the CNS, colloidal drug nanocarriers (i.e., micelles, liposomes, and nanoparticles) may be used that allow non-transportable drugs to cross the BBB by masking their intrinsic physicochemical characteristics through encapsulation of them inside nanocarriers. In recent years, endogenous transporters such as receptor transporters, carrier transporters and active efflux transporters, from the brain capillary endothelium have been used as ligands and are incorporated into the nanocarrier system to improve the delivery efficacy. The present invention encompasses pharmaceutical compositions prepared by these and other art-recognized techniques to deliver the oligonucleotide inhibitors of the present invention to the CNS.

In one embodiment, colloidal dispersion systems, such as macromolecule complexes, nanocapsules, microspheres, beads, and lipid-based systems including oil-in-water emulsions, micelles, mixed micelles, and liposomes, may be used as delivery vehicles for the oligonucleotide inhibitors of the present invention or constructs expressing them. Commercially available fat emulsions that may be suitable for delivering the nucleic acids of the invention include Intralipid®, Liposyn®, Liposyn® II, Liposyn® III, Nutrilipid, and other similar lipid emulsions. A preferred colloidal system for use as a delivery vehicle in vivo is a liposome (i.e., an artificial membrane vesicle). The preparation and use of such systems is well known in the art. Exemplary formulations are also disclosed in U.S. Pat. No. 5,981,505; U.S. Pat. No. 6,217,900; U.S. Pat. No. 6,383,512; U.S. Pat. No. 5,783,565; U.S. Pat. No. 7,202,227; U.S. Pat. No. 6,379,965; U.S. Pat. No. 6,127,170; U.S. Pat. No. 5,837,533; U.S. Pat. No. 6,747,014; and WO03/093449, which are herein incorporated by reference in their entireties.

In certain embodiments, liposomes used for delivery are amphoteric liposomes such SMARTICLES® (Marina Biotech, Inc.) which are described in detail in U.S. Pre-grant Publication No. 20110076322. The surface charge on the SMARTICLES® is fully reversible which make them particularly suitable for the delivery of nucleic acids. SMARTICLES® can be delivered via injection, remain stable, and aggregate free and cross cell membranes to deliver the nucleic acids.

In other embodiments, pharmaceutical compositions of the present invention may be administered directly to the central nervous system via intracerebroventricular (ICV) injection infusion into the cerebrospinal fluid (CSF), intrathecal injection, epidural injection, intraparenchymal infusion of the drug solution into the brain parenchyma using a catheter by convection-enhanced delivery (CED), and direct implantation of biodegradable drug delivery vehicles into brain parenchyma. Various routes of delivering an active agent to the CNS have been disclosed in an article entitled “Central Nervous System Drug Delivery” by C. Lei and C. Wang in the Journal of Controlled Release Topic Collection: Central Nervous System Drug Delivery Volume 2, Issue 2, which is incorporated by reference herein in its entirety.

One will generally desire to employ appropriate salts and buffers to render delivery vehicles stable and allow for uptake by target cells. Pharmaceutical compositions of the present invention comprise an effective amount of the delivery vehicle comprising the inhibitor polynucleotides (e.g., liposomes or other complexes or expression vectors) dissolved or dispersed in a pharmaceutically acceptable carrier or aqueous medium. The phrases “pharmaceutically acceptable” or “pharmacologically acceptable” refers to molecular entities and compositions that do not produce adverse, allergic, or other untoward reactions when administered to an animal or a human. As used herein, “pharmaceutically acceptable carrier” includes solvents, buffers, solutions, dispersion media, coatings, antibacterial and antifungal agents, isotonic and absorption delaying agents and the like acceptable for use in formulating pharmaceuticals, such as pharmaceuticals suitable for administration to humans. The use of such media and agents for pharmaceutically active substances is well known in the art. Except insofar as any conventional media or agent is incompatible with the active ingredients of the present invention, its use in therapeutic compositions is contemplated. Supplementary active ingredients also can be incorporated into the compositions, provided they do not inactivate the vectors or polynucleotides of the compositions.

The pharmaceutical forms suitable for injectable use include, for example, sterile aqueous solutions or dispersions and sterile powders for the extemporaneous preparation of sterile injectable solutions or dispersions. Generally, these preparations are sterile and fluid to the extent that easy injectability exists. Preparations should be stable under the conditions of manufacture and storage and should be preserved against the contaminating action of microorganisms, such as bacteria and fungi. Appropriate solvents or dispersion media may contain, for example, water, ethanol, polyol (for example, glycerol, propylene glycol, and liquid polyethylene glycol, and the like), suitable mixtures thereof, and vegetable oils. The proper fluidity can be maintained, for example, by the use of a coating, such as lecithin, by the maintenance of the required particle size in the case of dispersion and by the use of surfactants. The prevention of the action of microorganisms can be brought about by various antibacterial and antifungal agents, for example, parabens, chlorobutanol, phenol, sorbic acid, thimerosal, and the like. In many cases, it will be preferable to include isotonic agents, for example, sugars or sodium chloride. Prolonged absorption of the injectable compositions can be brought about by the use in the compositions of agents delaying absorption, for example, aluminum monostearate and gelatin.

Sterile injectable solutions may be prepared by incorporating the active compounds in an appropriate amount into a solvent along with any other ingredients (for example as enumerated above) as desired, followed by filtered sterilization. Generally, dispersions are prepared by incorporating the various sterilized active ingredients into a sterile vehicle which contains the basic dispersion medium and the desired other ingredients, e.g., as enumerated above. In the case of sterile powders for the preparation of sterile injectable solutions, the preferred methods of preparation include vacuum-drying and freeze-drying techniques which yield a powder of the active ingredient(s) plus any additional desired ingredient from a previously sterile-filtered solution thereof.

The compositions of the present invention generally may be formulated in a neutral or salt term. Pharmaceutically-acceptable salts include, for example, acid addition salts (formed with the free amino groups of the protein) derived from inorganic acids (e.g., hydrochloric or phosphoric acids), or from organic acids (e.g., acetic, oxalic, tartaric, mandelic, and the like). Salts formed with the free carboxyl groups of the protein can also be derived from inorganic bases (e.g., sodium, potassium, ammonium, calcium, or ferric hydroxides) or from organic bases (e.g., isopropylamine, trimethylamine, histidine, procaine and the like).

Upon formulation, compositions are preferably administered in a manner compatible with the dosage formulation and in such amount as is therapeutically effective. The formulations may easily be administered in a variety of dosage forms such as injectable solutions, oral extended release dosage forms and the like. For parenteral administration in an aqueous solution, for example, the solution generally is suitably buffered and the liquid diluent first rendered isotonic for example with sufficient saline or glucose. Such aqueous solutions may be used, for example, for intravenous, subcutaneous, and intradermal administration. Preferably, sterile aqueous media are employed as is known to those of skill in the art, particularly in light of the present disclosure. Some variation in dosage will necessarily occur depending on the condition of the subject being treated. The person responsible for administration will, in any event, determine the appropriate dose for the individual subject. Moreover, for human administration, preparations should meet sterility, pyrogenicity, general safety and purity standards as required by regulatory agencies.

In certain embodiments of the invention, the pharmaceutical compositions of the invention are packaged with or stored within a device for administration. Devices for injectable formulations include, but are not limited to, injection ports, autoinjectors, injection pumps, and injection pens. Devices for aerosolized or powder formulations include, but are not limited to, inhalers, insufflators, aspirators, and the like. Thus, the present invention includes administration devices comprising a pharmaceutical composition of the invention for treating or preventing one or more of the disorders described herein.

This invention is further illustrated by the following additional examples that should not be construed as limiting. Those of skill in the art should, in light of the present disclosure, appreciate that many changes can be made to the specific embodiments which are disclosed and still obtain a like or similar result without departing from the spirit and scope of the invention.

All patent and non-patent documents referenced throughout this disclosure are incorporated by reference herein in their entirety for all purposes.

### EXAMPLES

### Example 1: Effect of antimiR-155 Compounds on the Expression of Two Direct Seed-Matched Targets (Cux1 and CEBPB) in Human Monocytic Cells

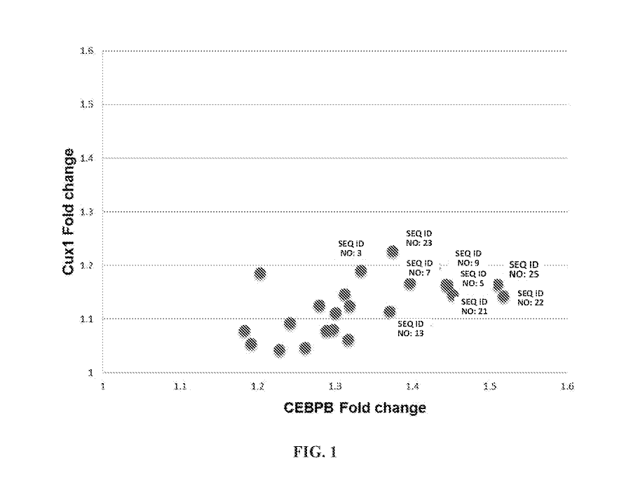

AntimiR-155 compounds were delivered to MV4-11 human monocytic cells by nucleofection. Regulation of 2 direct seed-matched targets (Cux1 and CEBPB) was measured by real-time PCR compared to untreated cells. The labeled compounds demonstrated the highest regulation of the 2 targets analyzed (FIG. 1).

### Example 2: Passive Uptake of antimiR-155 Compounds by Microglial Cells Up-Regulates the Expression of miR-155 Target Genes

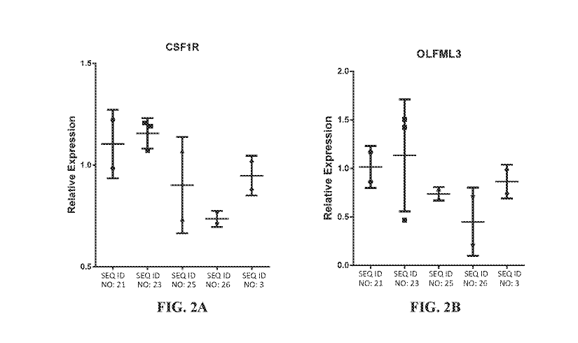

Microglia isolated from adult SOD1 mice were incubated passively in culture medium containing antitmiR-155 compounds (SEQ ID NOs: 21, 23, 25, 26, and 3) at 1 μM final concentration. Expression of 2 direct seed-matched targets (8-, 7-, and 6-nucleotide binding sites), CSF1R and OLFML3, was measured by real-time PCR in cells passively treated with antimiR-155 compounds and untreated cells (FIG. 2).

### Example 3: Administration of antimiR-155 Compounds into SOD1 Mice Up-Regulates the Expression of miR-155 Target Genes in Microglia

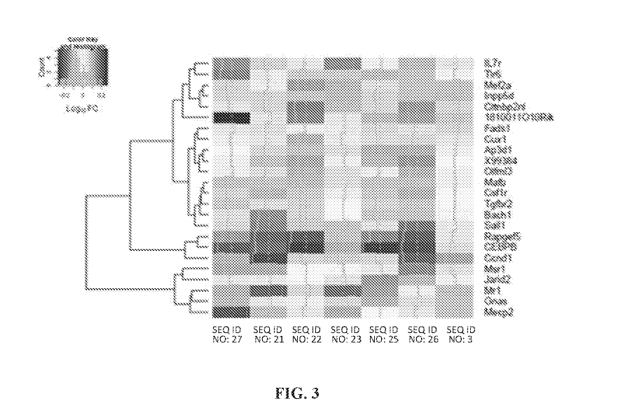

Six antimiR-155 compounds (SEQ ID NOs: 27, 21, 22, 23, 25, 26, and 3) were administered at a dose of 2 mg/kg via single intracerebroventricular (i.c.v.) injection into SOD1 mice (n=6-8 mice per treatment). Five days post-injection, microglia were isolated by cell sorting, and mRNA was harvested for gene expression by Nanostring codeset. A heatmap of the gene expression profile of predicted or validated miR-155 seed-matched targets is shown in FIG. 3. The heatmap shows the log 10 fold change versus saline for each gene in response to each antimiR-155 compound (average for all animals in a given treatment group). No statistical cuts were used for selection of this gene signature. AntimiR-155 compounds having SEQ ID NO: 23 and SEQ ID NO: 3 showed de-repression of the largest number of direct miR-155 targets.

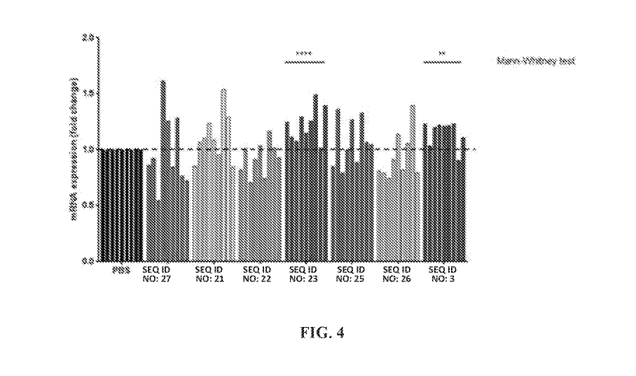

The Nanostring gene expression codeset was further analyzed and a set of direct target genes up-regulated in ≥4 mice by ≥2 antimiR-155 compounds was chosen to represent a gene expression signature for antimiR activity. FIG. 4 shows the fold-change results for this gene expression signature for each antimiR-155 compound. By Maim-Whitney non-parametric test, antimiR compounds with SEQ ID NOs: 23 and 3 showed a significant up-regulation of this set of targets.

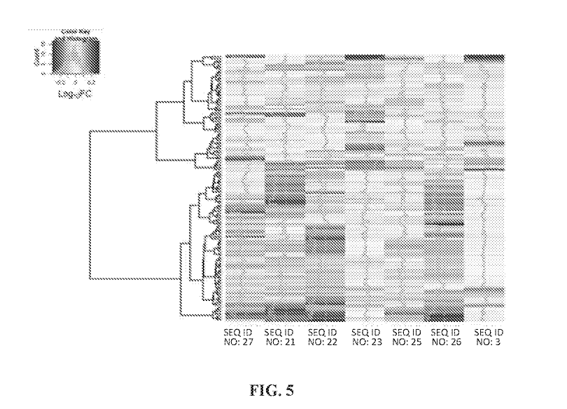

The Nanostring gene expression codeset was annotated for microglial homeostatic genes that are down-regulated in SOD1 mice but the expressions of which are restored to some extent in the miR-155 knock-out mouse. An average fold-change for each gene in this set versus saline was calculated for each antimiR-155 compound, and the log 10 fold-change was used to generate a heatmap (FIG. 5). AntimiR-155 compounds having SEQ ID NOs: 23 and 3 restored the expression of the highest number of genes in this gene set. AntimiR-155 compound having SEQ ID NO: 25 also showed a trend towards de-repression of these gene targets.

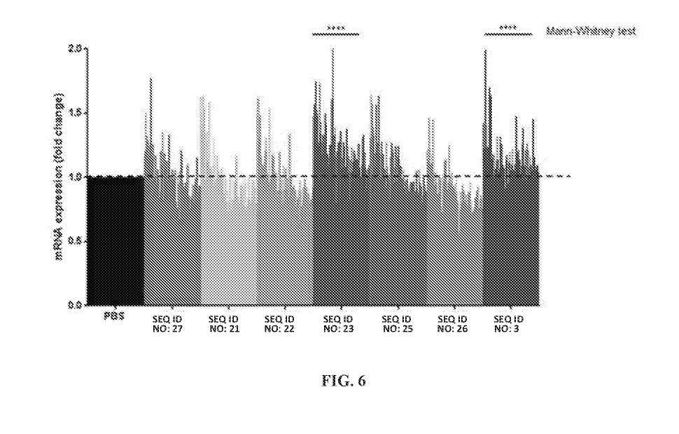

From the microglial homeostatic gene set described above, a set of direct target genes up-regulated in ≥4 mice by ≥2 antimiR-155 compounds was chosen to represent a gene expression signature for antimiR activity. FIG. 6 shows the fold-change results for this gene expression signature for each antimiR-155 compound. By Mann-Whitney non-parametric test, antimiR compounds with SEQ ID NOs: 23 and 3 showed a significant up-regulation of this set of homeostatic gene targets. The antimiR-155 compound having SEQ ID NO: 25 also showed a trend towards de-repression of this gene expression signature.

1. A method for treating a neurological disease in a subject in need thereof, comprising administering to the subject an oligonucleotide inhibitor of miR-155 comprising a sequence of 11 to 16 nucleotides, wherein
said oligonucleotide inhibitor is fully complementary to a mature sequence of miR-155 and has a full phosphorothioate backbone; and
wherein at least the first three nucleotides from the 3′ end of said oligonucleotide inhibitor are locked nucleotides and at least the second nucleotide from the 5′ end of the oligonucleotide inhibitor is a deoxyribonucleic acid (DNA) nucleotide.

2. The method of claim 1, wherein the neurological disease is amyotrophic lateral sclerosis (ALS).

3. The method of claim 1, wherein the sixth nucleotide from the 5′ end of said oligonucleotide inhibitor is a DNA nucleotide.

4. The method of claim 1, wherein the fourth nucleotide from the 3′ end of said oligonucleotide inhibitor is a locked nucleotide.

5. The method of any one of claims 1-4, wherein said oligonucleotide inhibitor contains at least nine locked nucleotides.

6. The method of any one of claims 1-5, wherein said oligonucleotide inhibitor has a length of 12 nucleotides.

7. The method of claim 1, wherein said oligonucleotide inhibitor has a sequence of SEQ ID NO: 23.

8. The method of any one of claims 1-7, wherein said oligonucleotide inhibitor upon administration reduces the activity or function of miR-155 in cells of the CNS.

9. The method of any one of claims 1-7, wherein said oligonucleotide inhibitor upon administration up-regulates the expression or activity of miR-155 target genes in cells of the CNS.

10. The method of any one of claims 1-7, wherein said oligonucleotide inhibitor upon administration up-regulates the expression or activity of homeostatic genes in cells of the CNS.

11. The method of any one of claims 1-7, wherein said oligonucleotide inhibitor upon administration reduces or inhibits the activity of inflammatory cells of the CNS.

12. The method of any one of claims 1-7, wherein said oligonucleotide inhibitor upon administration shows a reduction in the recruitment of inflammatory cells into the spinal cord.

13. The method of any one of claims 1-7, wherein said oligonucleotide inhibitor upon administration down-regulates the expression or activity of tissue-destructive genes and/or up-regulates the expression or activity of tissue-protective genes in cells of the CNS.

14. The method of any one of claims 8-13, wherein said inflammatory cells are selected from the group consisting of neuronal cells, monocytes, and microglia.

15. A method for reducing the activity of inflammatory cells in a neurological disease, comprising administering an oligonucleotide inhibitor of it miR-155 comprising a sequence of 11 to 16 nucleotides, wherein
said oligonucleotide inhibitor is fully complementary to a mature sequence of miR-155 and has a full phosphorothioate backbone; and
wherein at least the first three nucleotides from the 3′ end of said oligonucleotide inhibitor are locked nucleotides and at least the second nucleotide from the 5′ end of the oligonucleotide inhibitor is a deoxyribonucleic acid (DNA) nucleotide.

16. A method for treating or ameliorating neuro-inflammation in a subject in need thereof, comprising administering to the subject an oligonucleotide inhibitor of miR-155 comprising a sequence of 11 to 16 nucleotides, wherein
said oligonucleotide inhibitor is fully complementary to a mature sequence of miR-155 and has a full phosphorothioate backbone; and
wherein at least the first three nucleotides from the 3′ end of said oligonucleotide inhibitor are locked nucleotides and at least the second nucleotide from the 5′ end of the oligonucleotide inhibitor is a deoxyribonucleic acid (DNA) nucleotide.

17. The method of claim 16, wherein the subject in need thereof is suffering from or is at the risk of developing a neurological disease.

18. The method of any one of claim 15 or 16, wherein the neurological disease is amyotrophic lateral sclerosis (ALS).

19. The method of any one of claims 15-18, wherein the sixth nucleotide from the 5′ end of said oligonucleotide inhibitor is a DNA nucleotide.

20. The method of any one of claims 15-19, wherein e fourth nucleotide from the 3′ end of said oligonucleotide inhibitor is a locked nucleotide.

21. The method of any one of claims 15-20, wherein said oligonucleotide inhibitor contains at least nine locked nucleotides.

22. The method of any one of claims 15-21, wherein said oligonucleotide inhibitor has a length of 12 nucleotides.

23. The method of any one of claims 15-21, wherein said oligonucleotide inhibitor has a sequence of SEQ ID NO: 23.

24. The method of any one of claims 15-23, wherein said oligonucleotide inhibitor upon administration reduces the activity or function of mill-155 in cells of the CNS.

25. The method of any one of claims 15-23, wherein said oligonucleotide inhibitor upon administration up-regulates the expression or activity of miR-155 target genes in cells of the CNS.

26. The method of any one of claims 15-23, wherein said oligonucleotide inhibitor upon administration up-regulates the expression or activity of homeostatic genes in cells of the CNS.

27. The method of any one of claims 15-23, wherein said oligonucleotide inhibitor upon administration reduces or inhibits the activity of inflammatory cells of the CNS.

28. The method of any one of claims 15-23, wherein said oligonucleotide inhibitor upon administration shows a reduction in the recruitment of inflammatory cells into the spinal cord.

29. The method of any one of claims 15-23, wherein said oligonucleotide inhibitor upon administration down-regulates the expression or activity of tissue-destructive genes and/or up-regulates the expression or activity of tissue-protective genes in cells of the CNS.

30. The method of any one of claims 15-23, wherein said inflammatory cells are selected from the group consisting of neuronal cells, monocytes, and microglia.

31. A method for selecting a subject for treatment of a neurological disease, comprising determining a level of expression of one or more genes selected from the group consisting of IL7r, Tlr6, Mef2a, Inpp5d, Cttnbp2n1, 1810011O10Rik, Fads1, Cux1, Ap3d1, X99384, Olfm13, Mafb, Csf1r, Tgfbr2, Bach1, Sall1, Rapgef5, CEBPB, CCnd1, Msr1, Jarid2, Mr1, Gnas, and Mecp2, in CNS cells of the subject; comparing the level of the one or more genes in the CNS cells of the subject to a reference level of the one or more genes; and selecting a subject having an increase or a decrease in the level of the one or more genes in the CNS cells compared to the reference level for treatment of the neurological disease.
